(19) United States
(12) Patent Application Publication
(10) Pub. No.: US 2009/0230362 A1
(43) Pub. Date: Sep. 17, 2009

(54) CONJUGATED OLIGOELECTROLYTE ELECTRON TRANSPORTING LAYERS

(76) Inventors: Guillermo C. Bazan, Santa Barbara, CA (US); Yunhua Xu, Goleta, CA (US); Renqiang Yang, Qingdao City (CN); Thuc-Quyen Nguyen, Goleta, CA (US)

Correspondence Address: BERLINER & ASSOCIATES, 555 WEST FIFTH STREET, 31ST FLOOR, LOS ANGELES, CA, 90013, US

(21) Appl. No.: 12/359,967

(22) Filed: Jan. 26, 2009

Related U.S. Application Data
(60) Provisional application No. 61/062,409, filed on Jan. 25, 2008.

(51) Int. Cl. H01B 1/12; C07F 5/02; C07D 233/54

(52) U.S. Cl. 252/512; 548/110

(57) ABSTRACT

An organic electronic or an optoelectronic device containing a conjugated oligoelectrolyte. In more particularized embodiments, the conjugated oligoelectrolyte is the charge injection or transport layer. The conjugated oligoelectrolyte can be positively or negatively charged, and used in conjunction in a device with either or high or low work function metal.

### FIELD OF THE INVENTION

The invention relates to the use of a conjugated oligoelectrolyte in organic electronic devices; and more particularly as the charge injection/transport layer in polymer light emitting diodes.

### BACKGROUND OF THE INVENTION

Polymer LEDs (PLEDs) offer the opportunity of device fabrication using solution methods. However, multilayer fabrication is challenging if all the components display similar solubility characteristics. Depositing a new polymer layer can lead to removal of the underlying layer and/or mixing of the components. Conjugated polyelectrolytes are helpful in this context since their charged groups increase their solubility in polar solvents, such as water or methanol. The differences in solubility, compared to neutral conjugated polymers, are advantageous for fabricating the desired multilayer architectures.

Conjugated polymers with pendent ionic groups (conjugated polyelectrolytes, CPEs) or polar functionalities have gathered recent interest because they can function as very effective electron injection/transport layers (ETLs)¹ in polymer light emitting diodes (PLEDs) and thereby increase light output efficiencies. The mechanism of action remains under debate. One possibility involves lowering injection barriers by the introduction of permanent dipoles between the cathode and the semiconducting layer². The net result is the shift of the vacuum level and the lowering of the barrier heights for electron injection at the interface. Alternatively, in the case of CPEs, the applied field drives ion migration, which causes a redistribution of the internal field within the ETL³. Within this model, the device incorporates some of the characteristics of light emitting electrochemical cells (LECs)⁴. Despite these uncertainties, the use of CPEs opens the possibility of using high work function and stable metals, such as Al, Ag or Au, and the deposition of electrodes by printing techniques. All these considerations are relevant for developing white-light emitting devices based on organic electronic materials that may be more power efficient than conventional lighting sources. There is a need therefore for improved CPEs.

### BRIEF SUMMARY OF THE INVENTION

The present invention provides a new class of materials for use in organic electronic devices. In accordance with this invention, an organic electronic device is provided containing a conjugated oligoelectrolyte layer. In more particular embodiments, the conjugated electrolyte is the charge injection/transport layer. Oligomer species representative of conjugated oligoelectrolytes (COEs) show excellent function as ETL layers for use in displays and lighting devices. Relative to CPEs, COEs have better defined molecular structures, in that they are not described by molecular weight distributions, and can be obtained in higher degrees of purity by taking advantage of organic chemistry techniques. Additionally, because there are no batch-to-batch variations in molecular structure, one can obtain better reproducibility of device fabrication protocols. Unlike previous observations with their polymeric counterparts, the use of COEs leads to PLEDs that display fast temporal responses in their current densities and emission. These qualities provide substantial and unexpected improvements over prior devices.

More particularly, an organic electronic device such as, but not limited to, a light emitting diode; a light emitting electrochemical cell, or a photovoltaic device. Any structural modules from known conjugated polyelectrolytes can be extracted to make a conjugated oligoelectrolyte useful in this invention. They could contain a variety of conjugated repeat units, differently sized spacer units, molecular shapes, different types of cationic charges and charge compensating counterions. The charges on the oligoelectrolyte could also be anionic, or a combination of both cationic and anionic charges.

Compounds that can act as conjugated oligoelectrolytes in the present invention are, but not limited to, those containing any one or more, or any combination, of the following repeat units.

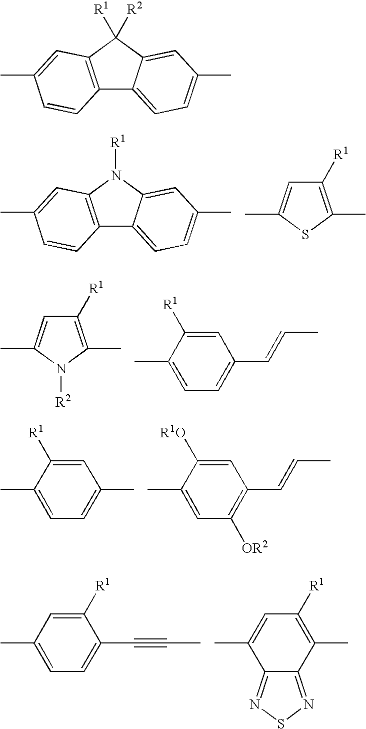

where R¹ and R² can be the same or different and where R¹ and R² can be either:

i) (CH₂)ₙN⁺BX⁻, where ‘B’ can be, but is not limited to, Me₃, Ethyl₃, Propyl₃, Me₂Ethyl, MeEthyl₂, Me₂Propyl, MePropyl₂, or Ethyl₂Propyl; ‘X’ can be, but is not limited to, Br, CF₃SO₃, BIm₄, BAr^{F}₄, BF₄, PF₆, F₃CSO₃, BPh₄, BTh₄, BPh^{F}₄, HPO₄²⁻ or FeCN₆⁴; and ‘n’ is an integer between 1 and 100 (or 1 and 50, or 1 and 20, or 1 and 10); or

ii) (CH₂)ₙZ, where ‘Z’ can be, but not limited to, SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻, or (NH₄)³⁺; ‘n’ is an integer between 1 and 100 (or 1 and 50, or 1 and 20, or 1 and 10).

More particularly, conjugated oligoelectrolytes can have any of the structures as disclosed in any of the FIGS. 10-13.

In preferred embodiments, the oligoelectrolyte is FFF-BIm₄, TriF-BIm₄, TetraF-BIm₄, HexaF-BIm₄, and FBF-SO₃Na, which are 9,9;9′,9′;9″,9″-Hexakis(6′″-N,N,N-trimethylammonium)hexyl)-2,2′;7′,2″-trifluorene tetrakis(1-imidazolyl)borate, 1,3,5-Tris(4-(9′,9′-bis(6″-N,N,N-trimethylammoniumhexyl)fluorene-2′-yl)phenyl)benzene tetrakis(1-imidazolyl)borate, Tetrakis(4-(2′-(9′,9′-bis(6″-N,N, N-trimethylammoniumhexyl))fluorenyl)phenyl)methane tetrakis(1-imidazolyl)borate, Hexakis(4-(2′-(9′,9′-bis(6″-N,N,N-trimethylammoniumhexyl))fluorenyl)phenyl)benzene tetrakis(1-imidazolyl)borate, and 1,4-Bis(9′,9′-di(4″-sulfonatobutyl)fluorene-2′-yl)benzene sodium, respectively.

In a specific, exemplary embodiment a cathode combination is provided comprising a conjugated oligoelectrolyte of the present invention, and a high work function metal. Such a high work function metal can be, but not limited to, aluminum (Al), gold (Au), silver (Ag) and copper (Cu). In a preferred example, the high work function metal is Al. Cathode combinations can also comprise of conjugated electrolytes with low work function metals, such as but not limited to barium (Ba), magnesium (Mg) and calcium (Ca).

### BRIEF DESCRIPTION OF THE DRAWINGS

For a more complete understanding of the present invention, reference is now made to the following descriptions taken in conjunction with the accompanying drawing, in which:

FIG. 1 shows the J-L-V characteristics of the LEDs ITO/PEDOT/MEH-PPV/ETL/Al, MEH-PPV/Al (circles), MEH-PPV/Ba/Al (squares), and MEH-PPV/FFF-BIm₄/Al, where FFF-BIm₄ is a COE of this invention.

FIG. 2 shows the time response curves of electroluminescence intensities at applied bias 4 V, MEH-PPV as emissive layer and FFF-BIm₄ as ETL (triangles), with the control device MEH-PPV/Ba/Al (circles).

FIG. 3 is an atomic force microscopy image of the FFF-BIm₄ layer as cast atop MEH-PPV.

FIG. 4 is the X-ray photoelectron spectroscopy spectra of COE FFF-Br (9,9;9′,9′;9″,9″-Hexakis(6′″-N,N,N-trimethylammonium)hexyl)-2,2′;7′,2″-trifluorene hexabromide.

FIG. 5 is the X-ray photoelectron spectroscopy spectra of COE FFF-BIm₄ and a high resolution region corresponding to N1s; and

FIG. 6 is a high resolution region corresponding to N1s of the spectra of FIG. 5.

FIG. 7 shows luminous efficiency vs. current density of various PLED devices, in which MEH-PPV is the emissive layer with and without FFF-BIm₄.

FIG. 8 shows the time response curves of current density at increased applied bias, MEH-PPV as emissive layer and FFF-BIm₄ as ETL, with the control device MEH-PPV/Ba/Al.

FIG. 9 shows the time response curves of luminance at increased applied bias, MEH-PPV as emissive layer and FFF-BIm₄ as ETL, with the control device MEH-PPV/Ba/Al.

FIG. 10 shows the structural formula of various oligoelectrolytes.

FIG. 11 shows the structural formula of various oligoelectrolytes.

FIG. 12 shows the structural formula of various oligoelectrolytes

FIG. 13 shows the structural formula of various oligoelectrolytes.

FIG. 14 shows a comparison of device performances; current density-voltage-luminance (J-V-L) (a) and luminous efficiency (b) with COE injection layers of TriF-BIm₄, TetraF-BIm₄, HexaF-BIm₄ and FPF-SO₃Na to those of devices with Ba/Al and Al as cathodes. TriF-BIm₄, TetraF-BIm₄, HexaF-BIm₄ are examples of cationic conjugated oligoelectrolytes with various molecular geometries, whereas FPF-SO₃Na is an example of an anionic conjugated oligoelectrolyte.

FIG. 15 shows the luminance-time characteristics of devices with COE injection layers of TriF-BIm₄, TetraF-BIm₄ and HexaF-BIm₄ and Ba/Al cathodes at 250 mA/cm².

FIG. 16 shows the Luminance-time characteristics of devices with COE injection layers of TriF-BIm₄, TetraF-BIm₄ and HexaF-BIm₄ and Ba/Al cathodes at 500 mA/cm².

FIG. 17 shows the luminance-time characteristics for (a) HexaF-BIm₄/Al and (b) Ba/Al devices at 250 mA/cm² after 10 minutes of thermal annealing.

FIG. 18 shows the luminance-time characteristics for HexaF-BIm4/Al and Ba/Al devices at 250 mA/cm² after 10 minute 80° C. annealing.

### DETAILED DESCRIPTION OF THE INVENTION

A new class of materials, conjugated oligoelectrolytes (COEs), is provided for use in organic electronic devices. In particular, these COEs can serve as the charge injection/transport layer (for example, materials for electron or hole injection/transport). The COEs show excellent function as ETL layers for use in displays and lighting devices. COEs have better defined molecular structures than current CPEs. COEs are not described by molecular weight distributions, and can be obtained in higher degrees of purity by taking advantage of organic chemistry techniques. Additionally, because there are no batch-to-batch variations in molecular structure, one can obtain better reproducibility of device fabrication protocols. The present invention provides PLEDs that display fast temporal responses in their current densities and emission.

The organic electronic device provided by this invention is exemplified by a light emitting diode, a light emitting electrochemical cell, a photovoltaic devices or similar devices that can incorporate the same class of organic semiconducting compounds. The term conjugated oligoelectrolytes as used herein refers to molecules that contain a well defined/discrete number of repeat units (where the spacer units can be the same or differently sized) and pendant groups bearing ionic functionalities. They may be linear, star shaped, tetrahedral, dimers, trimers, tetramers, pentamers, hexamers and analogous higher order homologs. The pendant groups could be cationic, anionic, or a combination of the two. These cationic or anionic charges allow the oligoelectrolyte to dissolve in polar solvents. However, not all conjugated units need to contain pendant ionic groups.

Compounds that can act as conjugated oligoelectrolytes in the present invention are, but not limited to, those containing one or more, or any combination of the following repeat units:

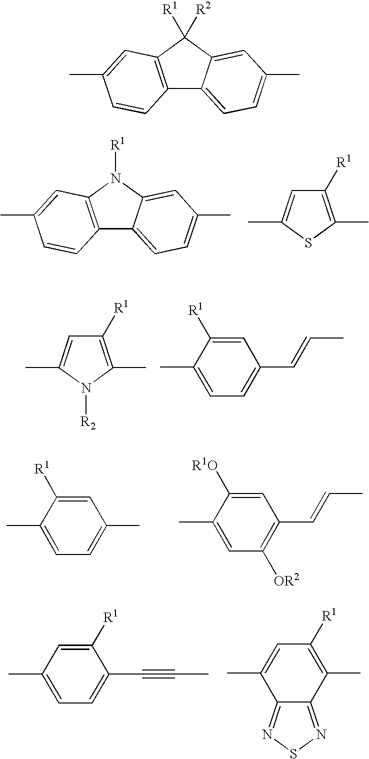

where R¹ and R² can be the same or different and where R¹ and R² can be either:

i) (CH₂)ₙN⁺BX⁻, where ‘B’ can be, but is not limited to, Me₃, Ethyl₃, Propyl₃, Me₂Ethyl, MeEthyl₂, Me₂Propyl, MePropyl₂, or Ethyl₂Propyl; ‘X’ can be, but is not limited to, Br, CF₃SO₃, BIm₄, BArF₄, BF₄, PF₆, F₃CSO₃, BPh₄, BTh₄, BPhF₄, HPO₄²⁻, or FeCN₆⁴; and ‘n’ is an integer between 1 and 100 (or 1 and 50, or 1 and 20, or 1 and 10); or

ii) (CH₂)ₙZ, where ‘Z’ can be, but not limited to, SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻, or (NH₄)³⁺; ‘n’ is an integer between 1 and 100 (or 1 and 50, or 1 and 20, or 1 and 10).

In other embodiments, the above repeatable units can form the basis of the following conjugated oligoelectrolytes:

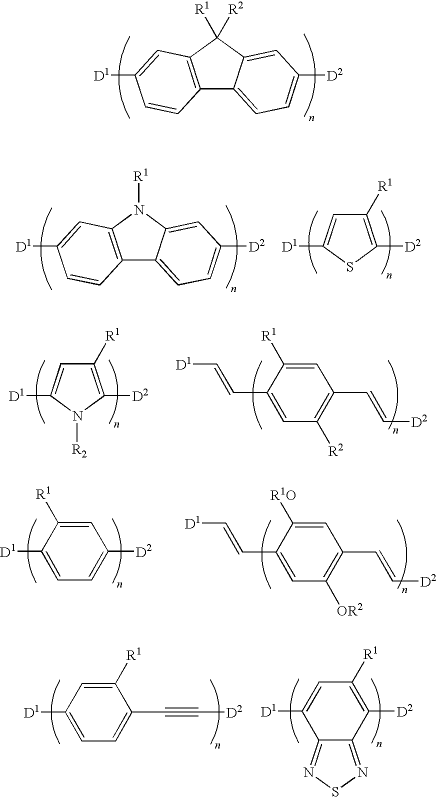

where ‘n’ is any integer between 1 and 100; 1 and 50; 1 and 20; or more preferably 1 and 10; and where terminal end groups D¹ and D² are the same or different and can be a hydrogen, alkyl group, alkoxy group or any one of the following:

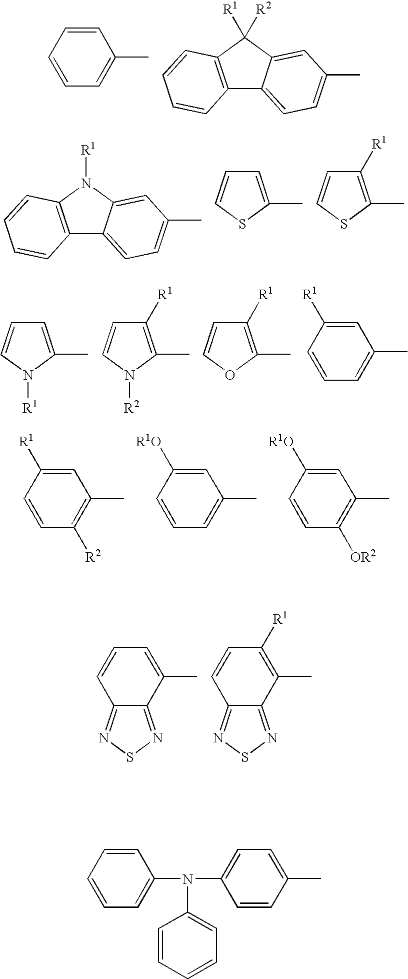

and where R¹ and R² can be the same or different and where R¹ and R² can be either:

i) (CH₂)ₙN⁺BX⁻, where ‘B’ can be, but is not limited to, Me₃, Ethyl₃, Propyl₃, Me₂Ethyl, MeEthyl₂, Me₂Propyl, MePropyl₂, or Ethyl₂Propyl; ‘X’ can be, but is not limited to, Br, CF₃SO₃, BIm₄, BArF₄, BF₄, PF₆, F₃CSO₃, BPh₄, BTh₄, BPh^{F}₄, HPO₄²⁻, or FeCN₆⁴; and ‘n’ is an integer between 1 and 100 (or 1 and 50, or 1 and 20, or 1 and 10); or

ii) (CH₂)ₙZ, where ‘Z’ can be, but not limited to, SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻, or (NH₄)³⁺; ‘n’ is an integer between 1 and 100 (or 1 and 50, or 1 and 20, or 1 and 10).

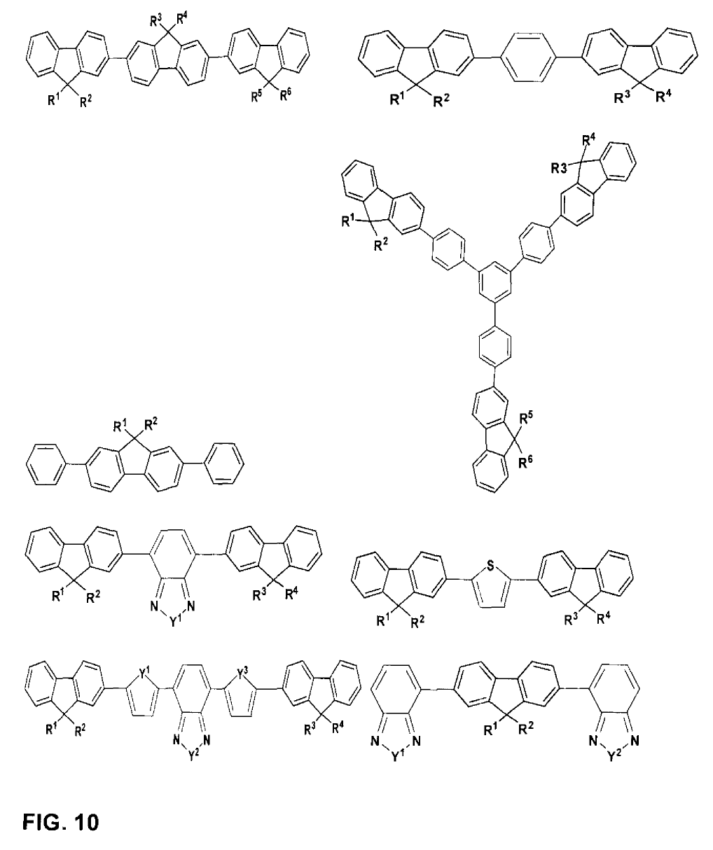

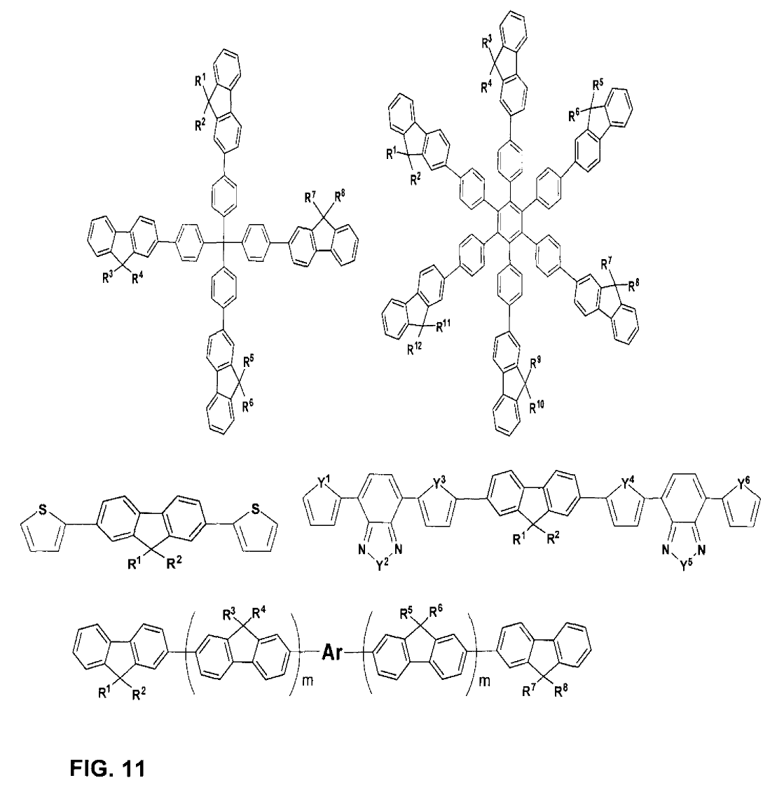

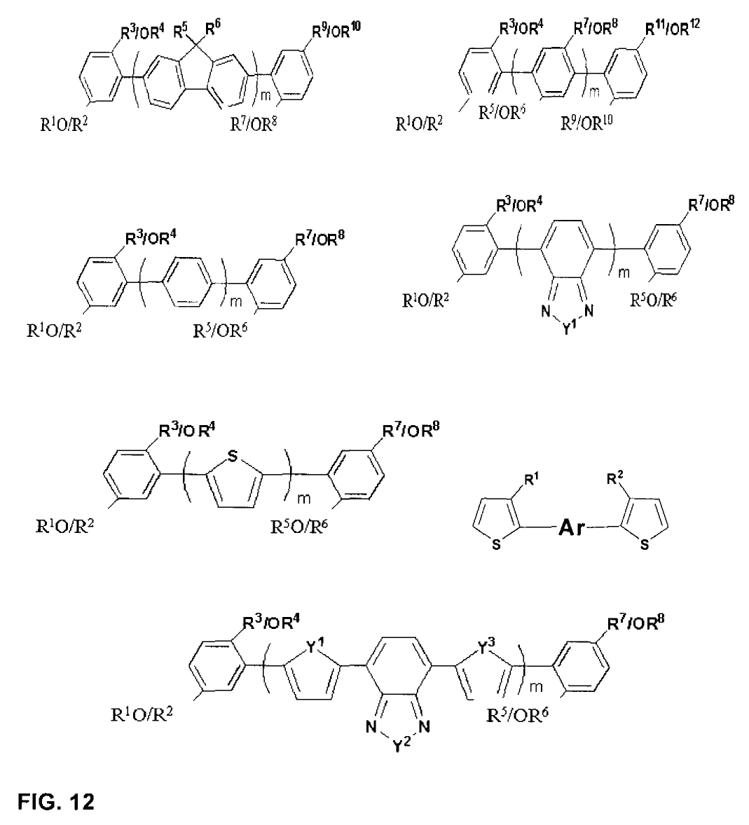

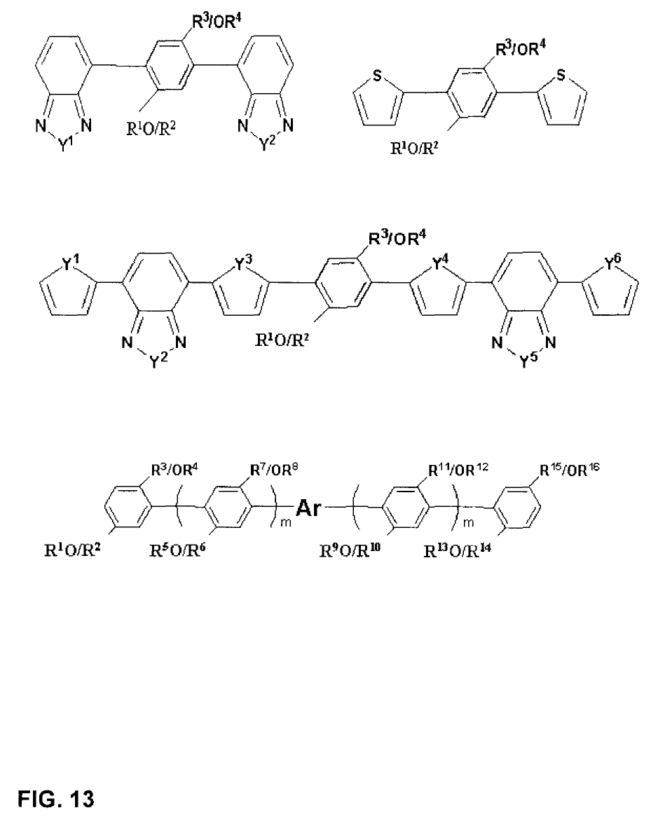

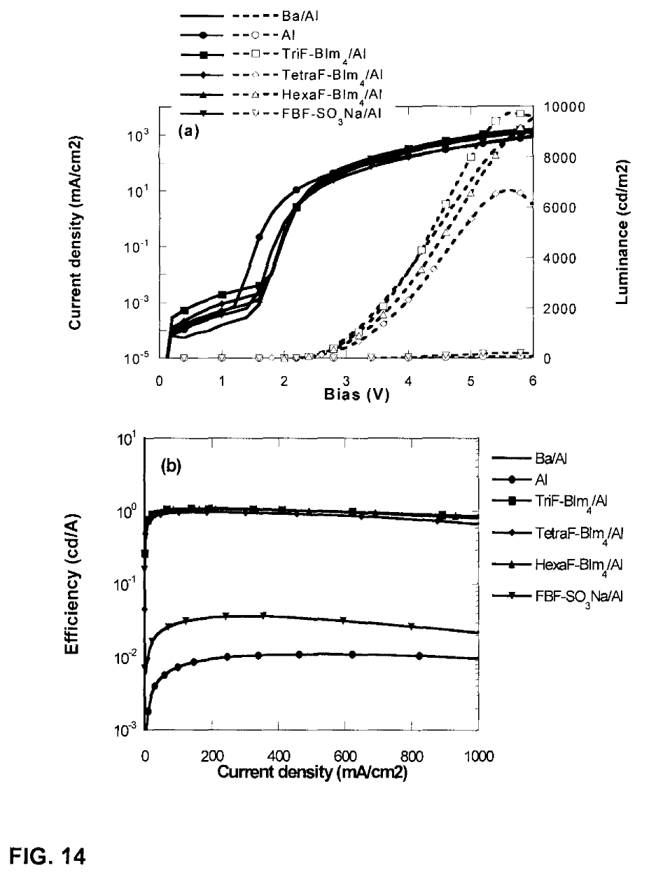

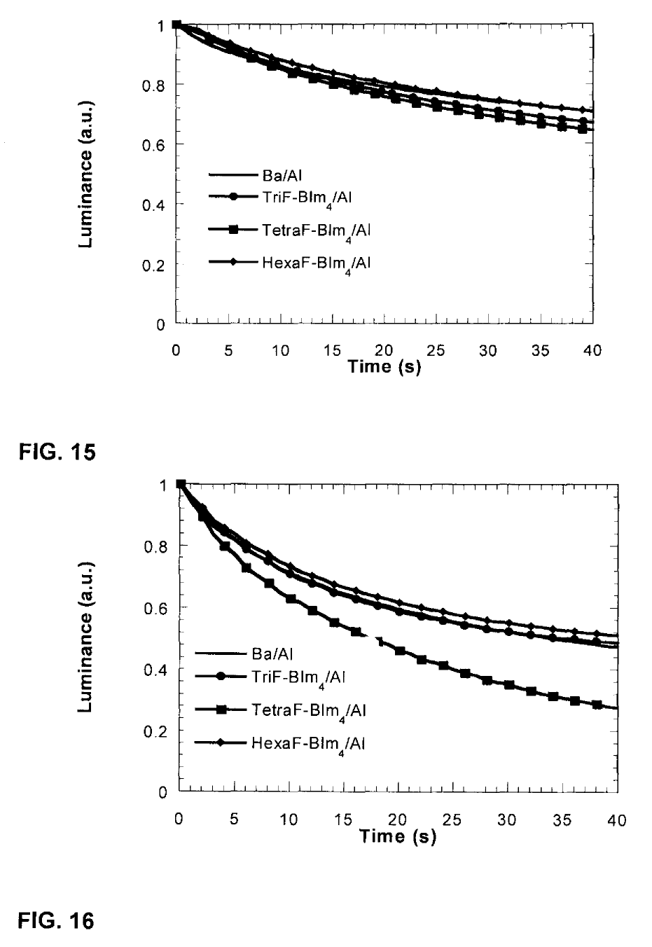

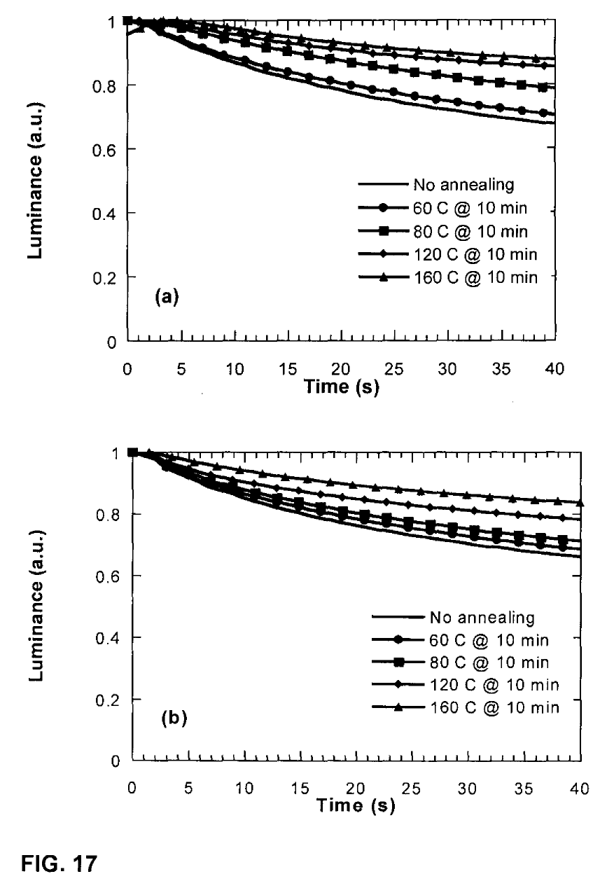

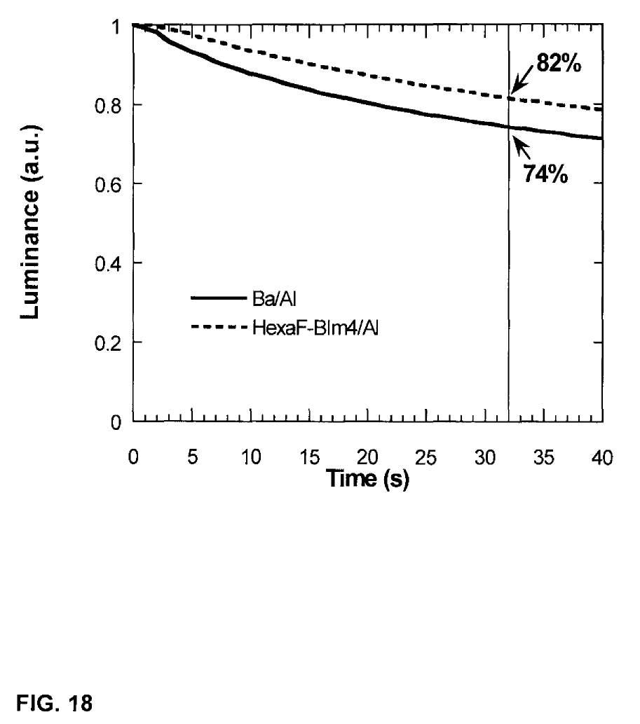

FIGS. 10-13 disclose more particular conjugated oligoelectrolytes of the present invention. These oligoelectrolytes can possess one or more ‘Rs’ (R¹, R², etc) groups which can be the same or different, where ‘Rs’ can be (CH₂)ₙN⁺BX⁻, where ‘n’ and ‘m’ are integers between 1 and 100 (or 1 and 50, or 1 and 20, or 1 and 10); ‘B’ can be, but is not limited to, Me₃, Ethyl₃, Propyl₃, Me₂Ethyl, MeEthyl₂, Me₂Propyl, MePropyl₂, or Ethyl₂Propyl; ‘X’ can be, but is not limited to, Br, CF₃SO₃, BIm₄, BAr^{F}₄, BF₄, PF₆, F₃CSO₃, BPh₄, BTh₄, BPhF₄, HPO₄²⁻, or FeCN₆⁴; ‘Y¹’ to ‘Y⁶’ is O, S, Se, or N—R′, and where ‘Y¹’ to ‘Y⁶’ can be the same or different; and wherein ‘Ar’ is one of the following

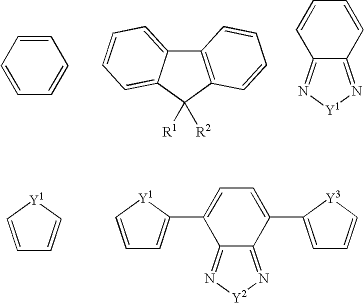

In more particular embodiments, the above oligoelectrolytes can have an ‘n’ equal to 6, ‘B’ is Me₃ and X′ is BIm₄.

In other embodiments the oligoelectrolytes, such as those disclosed in FIGS. 10-13, can possess one or more ‘Rs’ (i.e. R¹, R² etc.) groups which can be the same or different, and where these ‘Rs’ can be (CH₂)ₙZ, where ‘Z’ can be, but not limited to, SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻, or (NH₄)³⁺; ‘n’ and ‘m’ are integers between 1 and 100 (or 1-50, or 1-20, or 1-10); ‘Y¹’ to ‘Y⁶’ is O, S, Se, or N—R′, and where ‘Y¹’ to ‘Y⁶’ can be the same or different; and wherein ‘Ar’ is one of the following

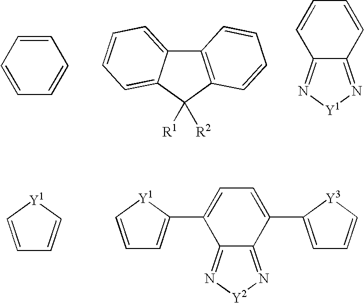

In more particular embodiments, ‘n’ is 4, and ‘Z’ is SO₃⁻Na⁺.

In preferred embodiments, the oligoelectrolyte is FFF-BIm₄, TriF-BIm₄, TetraF-BIm₄, HexaF-BIm₄, and FBF-SO₃Na, which are 9,9;9′,9′;9″,9″-Hexakis(6′″-N,N,N-trimethylammonium)hexyl)-2,2′,7′,2″-trifluorene tetrakis(1-imidazolyl)borate, 1,3,5-Tris(4-(9′,9′-bis(6″-N,N, N-trimethylammoniumhexyl)fluorene-2′-yl)phenyl)benzene tetrakis(1-imidazolyl)borate, Tetrakis(4-(2′-(9′,9′-bis(6″-N,N, N-trimethylammoniumhexyl))fluorenyl)phenyl)methane tetrakis(1-imidazolyl)borate, Hexakis(4-(2′-(9′,9′-bis(6″-N,N,N-trimethylammoniumhexyl))fluorenyl)phenyl)benzene tetrakis(1-imidazolyl)borate, and 1,4-Bis(9′,9′-di(4″-sulfonatobutyl)fluorene-2′-yl)benzene sodium, respectively.

In a specific, exemplary embodiment a cathode combination is provided comprising a conjugated oligoelectrolyte of the present invention, and a high work function metal, such as, but not limited to, Al, Au, Ag and Cu. In a preferred example, the high work function metal is aluminum Al. Cathode combinations can also comprise of conjugated electrolytes with low work function metals, such as but not limited to Ba and Ca.

Shown in Scheme 1 is the synthesis and structure of a hexacationic fluorene trimer bearing (N,N,N-trimethylammonium)hexyl substituents at the 9 positions and tetrakis(1-imidazolyl)borate counterions (FFF-BIm₄). Conditions: i) Pd(PPh₃)₄, 2 M Na₂CO₃, toluene, reflux, 24 h, ii) NMe₃, THF/methanol, iii) NaBIm₄, water, dialysis. This molecule is representative of three consecutive repeat units in the corresponding cationic poly(fluorene) species⁵. The choice of the BIm₄ anion was made on the basis of previous excellent ETL performance by cationic CPEs with this counterion⁶. The overall synthesis involves a standard Pd-mediated Suzuki cross coupling reaction of 2,7-bis(4,4,5,5-tetramethyl-1,3,2-dioxaborolan-2-yl)-9,9-bis(6′-bromohexyl)fluorene with 2-bromo-9,9-bis(6′-bromohexyl)fluorene to form the conjugated core (step i in Scheme 1). The resulting neutral fluorene trimer can be purified by standard chromatography and was analyzed by NMR spectroscopy, mass spectrometry and elemental analysis. Quaternization of the neutral species by using trimethylamine in a THF/methanol solvent mixture (step ii), followed ion exchange of bromide using NaBIm₄ by dialysis (step iii) yields FFF-BIm₄ as a white powder. The overall yield of the sequence of reactions is excellent, approximately 75%. XPS analysis confirmed quantitative exchange of bromide for (BIm₄)⁻. FFF-BIm₄ is soluble in polar solvents (methanol, water, DMF, DMSO, etc.). It would be apparent to persons of skill in the art that conjugated electrolytes other than FFF-BIm₄ can be made using similar procedures.

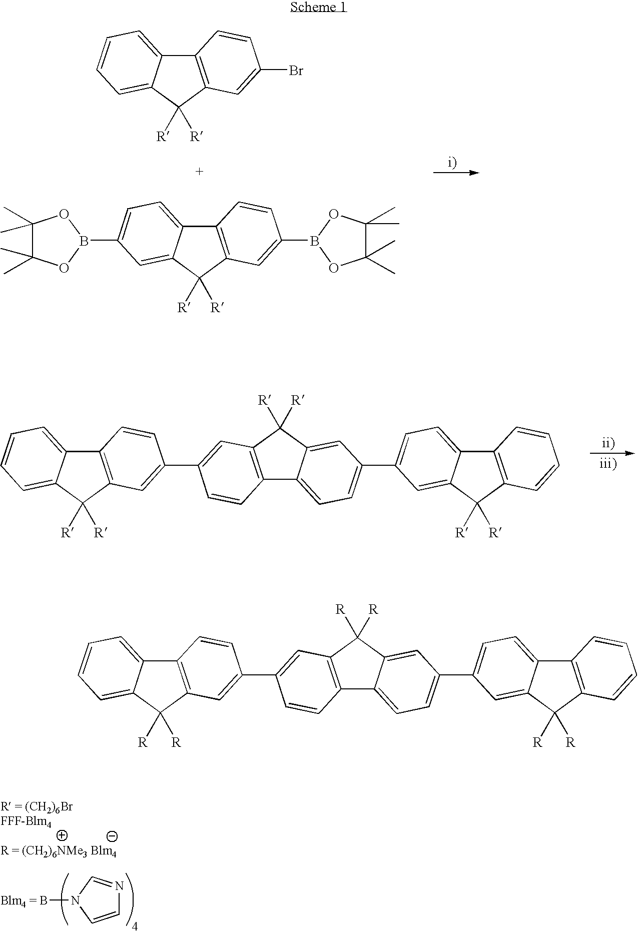

The PLED test devices used for our measurements had the general architecture ITO/PEDOT:PSS/MEH-PPV/FFF-BIm₄/Al (PEDOT: poly(ethylenedioxythiophene):poly(styrenesulfonic acid), MEH-PPV: poly(2-methoxy-5-(2′-ethylhexyloxy)-1,4-phenylenevinylene). Fabrication followed closely the procedures used for devices containing a CPE ETL⁶. All the steps were carried out by spin coating the films from solution, except for the cathode deposition (Al or Ba/Al), which was accomplished by thermal evaporation. The most noteworthy step in the overall process was the spin coating of FFF-BIm₄ from a methanol solution atop MEH-PPV. No intermixing of the FFF-BIm₄ and MEH-PPV layers was anticipated because of the orthogonal solubility of the two materials.^{7,8,9} Such procedures could be adapted to fabricate devices with other COEs as disclosed in this application.

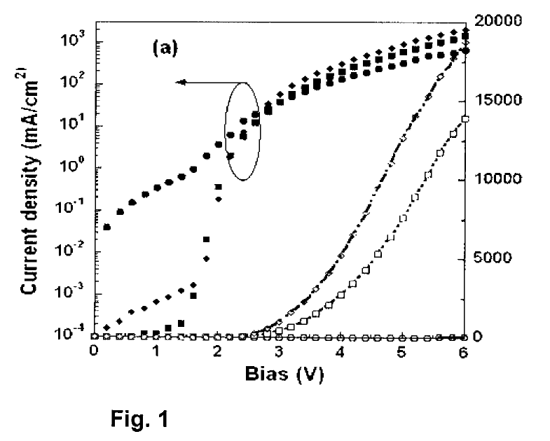

Shown in FIG. 1 are the current density-luminance-voltage (J-L-V) characteristics of the PLEDs. These results were obtained by testing inside a nitrogen filled glovebox. Also included in FIG. 1 are data for PLEDs with structures ITO/PEDOT:PSS/MEH-PPV/Ba/Al and ITO/PEDOT:PSS/MEH-PPV/Al. The former allows comparison against a device where ohmic contact takes place between the cathode and MEH-PPV, while the latter provides insight into the effect of the ETL. One observes in FIG. 1 that the insertion of the FFF-BIm₄ ETL gives rise to J and L that are higher than that obtained with Ba. In these devices, the emission arises solely from the MEH-PPV layer. Furthermore, there is no emission in the case of ITO/PEDOT/MEH-PPV/Al, consistent with poorly balanced charge injection and therefore ineffective electron injection Examination of the luminous efficiency (cd/A) vs. J shows that the FFF-BIm₄/Al device is most efficient. For example, the cd/A observed at 200 mA/cm² are 1.66 (3.7 V), FFF-BIm4/Al; 1.32 (4.0 V), Ba/Al and 0.006 (4.4 V), Al. In particular, the excellent performance obtained by combination of a COE and a high work function metal is thus demonstrated.

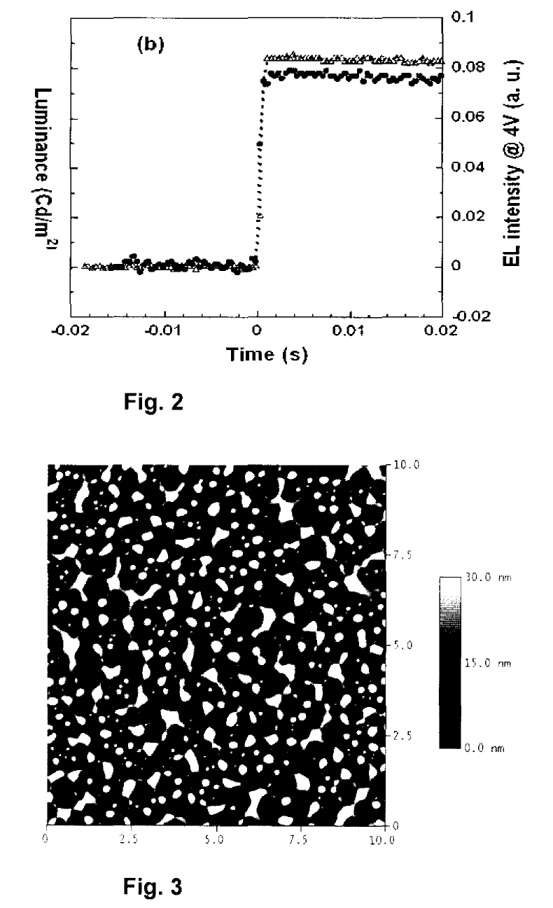

Introduction of CPE ETLs can lead to PLEDs with temporal responses of the current density and luminance that are in the order of seconds. Such a timescale is consistent with ion migration mediating device performance and, in particular, the electron injection barrier. Shown in FIG. 2 are the electroluminescence (EL) intensities vs. time plots for the ITO/PEDOT/MEH-PPV/FFF-BIm₄/Al and ITO/PEDOT/MEH-PPV/Ba/Al devices within a time domain of less than 20 msec. These measurements involved applying a rectangular voltage pulse to the device. The EL response was registered by a Si photodiode in photovoltaic regime connected to an oscilloscope. Photocurrent traces were subsequently digitized by the oscilloscope. FIG. 2 shows that, at least within the resolution available with our instrumentation, it was not possible to distinguish any temporal differences between the two devices. Thus, the response of the device when using FFF-BIm₄ is considerably faster than that observed when using a CPE ETL. With the latter materials it is possible to observe a response time (defined as the time when J is 50% of its maximum value) of 20 seconds at 4 V³.

Atomic force microscopy (AFM) was used to examine the quality of the FFF-BIm₄ layers. Our main concern was the possibility of crystallization leading to rough surface features because FFF-BIm₄ is an ionic molecule of intermediate dimensions. FIG. 3 shows the surface topography of the FFF-BIm₄ layer cast atop the MEH-PPV film under conditions identical to device fabrication. Indeed, the surface is very rough. However, as shown by the device data shown above, this non-homogenous film layer does not impact negatively the device performance. This is a significant departure from the prior art. Part of the reason why COEs have not been used before is that they do not lead to nice “perfect looking” films. However, as we show here, forming a good layer after spin coating is not important, a finding that will not be obvious to practitioners of the art. It should be noted that similar observations are obtained with the other COE structures provided in this application.

Accordingly, we have shown that COEs can function very well as the ETL in PLEDs. Indeed, Ohmic-type contacts can be obtained using cathodes made from Aluminum, a high work function, cheap and stable metal. The response time of the PLEDs is faster than that observed with CPEs. Finally, we note that smooth ETL layers are not a prerequisite for device function. The better structural and molecular control afforded by the COE indicates that they are superior materials for ETLs, compared with their polymeric counterparts.

The following Examples will provide details for the synthesis and structure of an exemplary conjugated oligoelectrolyte of this invention, specifically, a hexacationic fluorene trimer bearing (N,N,N-trimethylammonium)hexyl substituents at the 9 positions and tetrakis(1-imidazolyl)borate counterions (FFF-BIm₄) as well as the fabrication of a light emitting Diode containing the conjugated oligoelectrolyte as a charge injection/transport layer of a light emitting diode.

### Example 1

### Synthesis of FFF-BIm₄

All commercial chemical reagents were obtained from Aldrich and used as received. The ¹H and ¹³C NMR spectra were collected on a Varian ASM-100 400 MHz spectrometer.

2-Bromo-9,9-bis(6′-bromohexyl)fluorene (1). To a 150 ml of 45% aqueous potassium hydroxide was added 6.0 g (24.4 mmol) 2-bromofluorene, 59.3 g (0.244 mol) 1,6-dibromohexane and 0.78 g (2.4 mmol) tetrabutylammonium bromide at 75° C. The mixture was stirring for one hour, and then cooled down to room temperature. The aqueous layer was extracted with dichloromethane. The organic layer was washed with 1.0 M aqueous HCl, then brine and water, and dried over anhydrous magnesium sulfate. After removal of the solvent and the excess 1,6-dibromohexane under reduced pressure, the residue was purified by column chromatography on silica gel (eluent CH₂Cl₂/hexanes, 1:15) to give white oil product 12.0 g (86%). ¹H NMR (400 MHz, CDCl₃). δ (ppm): 7.68 (br, 1H), 7.57 (br, 1H), 7.48 (d, 1H), 7.46 (d, 1H), 7.34 (br, 3H), 3.29 (t, 4H, J=6.8 Hz), 1.96 (m, 4H), 1.67 (q, 4H, J=6.8 Hz), 1.20 (m, 4H), 1.08 (m, 4H), 0.60 (m, 4H). ¹³C NMR (100 MHz, CDCl₃). δ (ppm): 152.76, 150.09, 140.33, 140.21, 130.23, 127.78, 127.28, 126.22, 122.97, 121.32, 121.23, 120.03, 55.43, 40.33, 34.17, 32.80, 29.19, 27.95, 23.65.

9,9;9′,9′;9″,9″-Hexakis(6′″-bromohexyl)-2,2′;7′,2″-trifluorene (2). A mixture of 2,7-bis(4,4,5,5-tetramethyl-1,3,2-dioxaborolan-2-yl)-9,9-bis(6′-bromohexyl)fluorene (0.5 mmol, 372 mg), 2-bromo-9,9-bis(6′-bromohexyl)fluorene (1.075 mmol, 614 mg), Pd(PPh₃)₄ (15 mg) were dissolved in 8 mL toluene and 2.5 mL 2 M Na₂CO₃ aqueous solution (Scheme 2). The solution was refluxed at stirring for two days, and then cooled to room temperature and diluted with 150 mL of dichloromethane. The organic layer was collected, washed with brine and water, and then dried over sodium sulfate. After evaporation the solvent, the residue was purified by chromatograph on silicon gel with the eluent hexanes: CH₂Cl₂=2:1, to give white solid compound 508 mg (69%). ¹H NMR (400 MHz, CDCl₃). δ (ppm): 7.83 (q, 4H), 7.76 (br, 2H), 7.70 (m, 4H), 7.65 (d, 4H), 7.37 (m, 6H), 3.29 (t, 12H, J=6.8 Hz), 2.10 (br, 12H), 1.69 (m, 12H), 1.24 (m, 12H), 1.13 (m, 12H), 0.75 (br, 12H). ¹³C NMR (100 MHz, CDCl₃), δ (ppm): 151.63, 151.36, 150.78, 140.96, 140.64, 140.60, 140.24, 127.38, 127.19, 126.52, 126.41, 123.05, 121.48, 121.39, 120.31, 120.25, 120.05, 55.47, 55.29, 40.46, 34.25, 32.81, 29.25, 27.96, 23.86, 23.78.

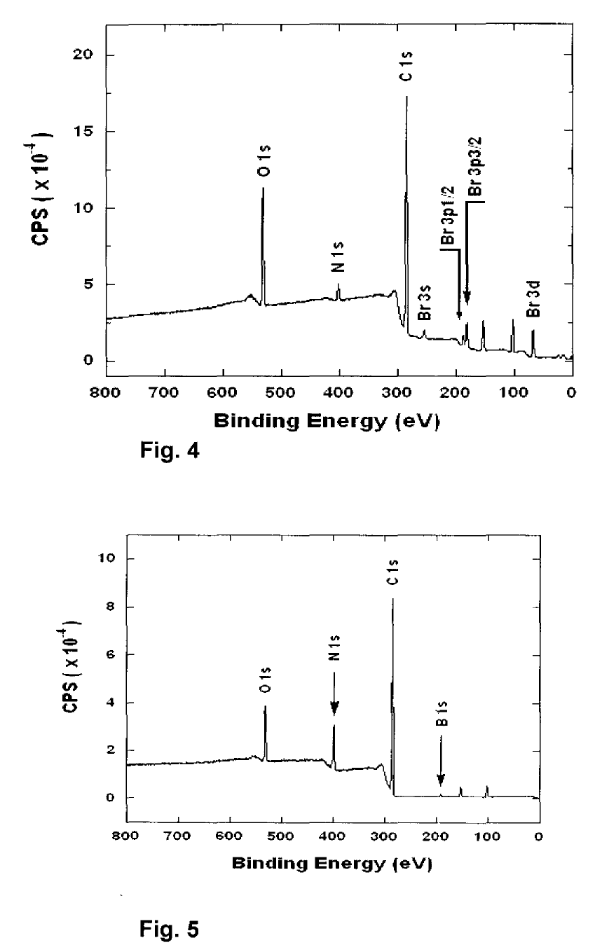

9,9;9′,9′;9″,9″-Hexakis(6′″-N,N,N-trimethylammonium)hexyl)-2,2′;7′,2″-trifluorene hexabromide (3, FFF-Br). Condensed trimethylamine (2.5 ml) was added dropwise to a solution of neutral oligomer 2 (147 mg, 0.1 mmol) in tetrahydrofurnan (20 ml) at −78° C. The mixture was allowed to warm up to room temperature gradually. After stirring 24 h, some precipitate was redissolved by addition of excess methanol, and then extra 2 ml trimethylamine was added at −78° C., and the mixture was stirred vigorous for 24 h at room temperature. After removal of the solvents, hexanes and acetone were added to wash the residue respectively, and then collected, drying in vacuum oven to give the target oligomer 144 mg (79%) as white solid. ¹H NMR (400 MHz, CD₃OD). δ (ppm): 7.88 (q, 4H), 7.75 (m, 10H), 7.44 (m, 2H), 7.34 (br, 4H), 3.22 (m, 12H), 3.03 (s, 54H), 2.18 (br, 12H), 1.58 (br, 12H), 1.16 (br, 24H), 0.68 (br, 12H). ¹³C NMR (100 MHz, CD₃₀D), δ (ppm): 153.00, 152.63, 152.02, 142.43, 142.19, 141.81, 141.73, 128.57, 128.39, 127.56, 127.46, 124.24, 122.29, 121.57, 121.46, 121.06, 67.74, 56.84, 56.56, 53.66, 41.47, 41.34, 30.59, 30.44, 27.14, 27.03, 25.21, 24.97, 23.93, 23.81. XPS spectra, FIG. 4.

General procedure for counterion exchange. 9,9;9′,9′;9′,9″-Hexakis(6′″-N,N,N-trimethylammonium)hexyl)-2,2′;7′,2″-trifluorene bromide (FFF-Br) (91.3 mg, 0.05 mmol) was dissolved in 5 ml methanol (Scheme 1). Subsequently, a solution of excess interested salt in 8 ml water was added. After stirring for half an hour, then the mixture was poured into membrane to remove the excess small salts. The water was evaporated under reduced pressure after dialysis three days. Finally the residue was dried under vacuum. This dialysis process for the counterion exchange of the oligomer was verified to be successful after characterization of the obtained products.

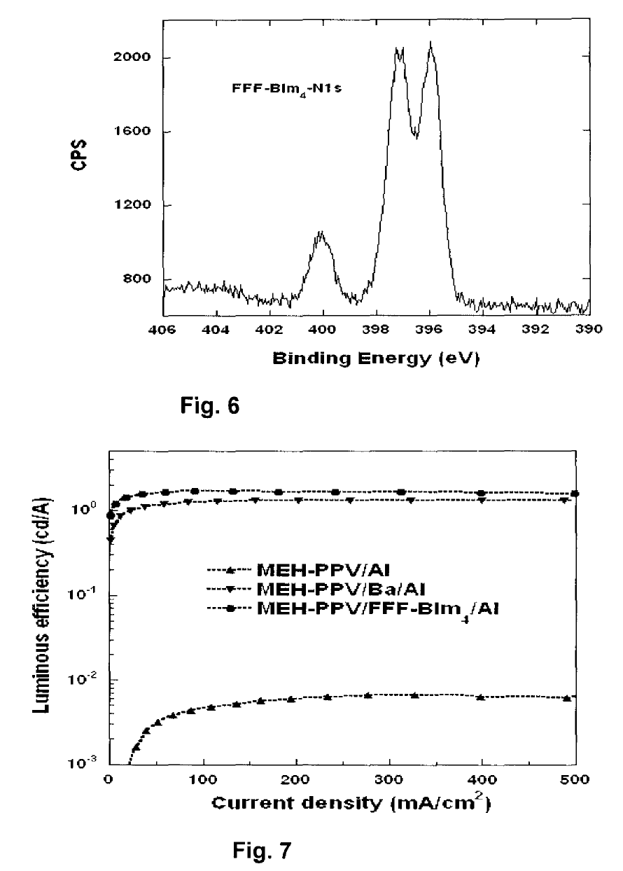

9,9;9′,9′;9″,9″-Hexakis(6′″-N,N,N-trimethylammonium)hexyl)-2,2′;7′,2″-trifluorene tetrakis(1-imidazolyl)borate (FFF-BIm₄). FFF-Br 91.3 mg (0.05 mmol) and sodium tetrakis(1-imidazolyl)borate 272 mg (0.90 mmol) were used, and obtained target oligomer 83 mg (55%). ¹H NMR (400 MHz, CD₃OD). δ (ppm): 7.89 (q, 4H), 7.74 (m, 10H), 7.46 (m, 2H), 7.34 (br, 4H), 7.11 (s, 24H), 6.96 (s, 24H), 6.76 (s, 24H), 3.20 (m, 12H), 3.02 (s, 54H), 2.17 (br, 12H), 1.56 (br, 12H), 1.14 (br, 24H), 0.68 (br, 12H). XPS spectra, FIGS. 5 and 6.

### Example 2

### Light-Emitting Diode Fabrication

Devices were fabricated on pre-patterned indium-tin oxide (ITO) with sheet resistance 10-20Ω/□. The substrate was cleaned under ultrasonic conditions with detergent, de-ionized water, acetone and isopropanol. An oxygen plasma treatment was made for 20 minutes as the final step of substrate cleaning procedure. On the top of ITO glass a layer of polyethylenedioxythiophene:polystyrene sulfonic acid (PEDOT:PSS) film with thickness of 50 nm was spin-coated from its aqueous dispersion (Baytron P 4083, Bayer AG.), aiming to improve the hole injection and to avoid the possibility of leakage. PEDOT:PSS film was dried at 120° C. for 2 hours in the vacuum oven. The solution of emissive material, poly[2-methoxy-5-(2′-ethylhexyloxy)-1,4-phenylenevinylene] (MEH-PPV) in toluene, was spin-coated on top of ITO/PEDOT:PSS surface as emissive layer, which typical thickness was 70-80 nm. Then a thin layer of oligomer electrolytes FFF-BIm₄ (Scheme 1) as an electron transporting layer (ETL) was spin-coated from its methanol solution, and the thickness was about 10 nm. The interface of the two layers was not mixed because of orthogonal polarity of solvents. The subsequent 200 nm thick aluminum capping layer was thermally deposited by vacuum evaporation through a mask at a base pressure below 2×10⁻⁴ Pa. The spin coating of EL layers and the device performance tests were carried out within a glove box with nitrogen circulation. At the same batch, the control devices were fabricated and tested with the following configuration, ITO/PEDOT/MEH-PPV/Ba/Al and ITO/PEDOT/MEH-PPV/Al, respectively. Current-voltage (J-V) characteristics were measured with a computerized Keithley 236 Source Measure Unit.

Referring to FIG. 7, the luminous efficiency of these devices was examined. Highly significant is that the efficiency of the device with FFF-BIm₄ as ETL is higher than that of the Ba/Al device.

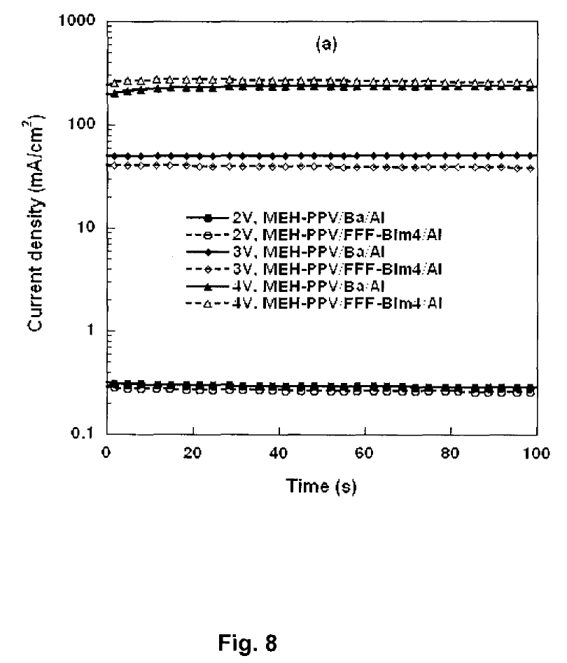

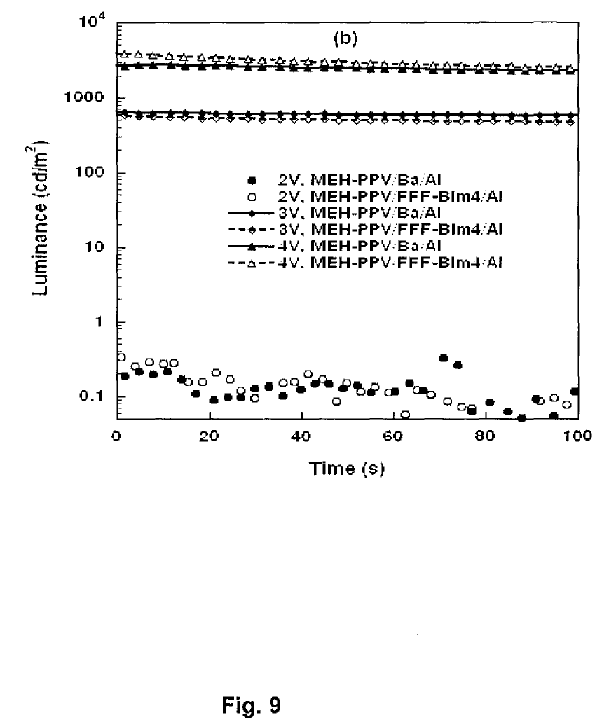

Referring to FIG. 8, the time response of the devices, MEH-PPV/Ba/Al and MEH-PPV/FFF-BIm₄/Al, were measured at increased applied bias. One can observe that the current density time response curve of FFF-BIm₄/Al device was a little lower than that of Ba/Al at lower applied voltages (2 V and 3 V). However, and referring to FIG. 9, when increasing the bias to 4 V, the curves were reversed. Consistently, their luminance time response curves keep the same trend.

Table 1 shows the performance of the PLEDs:

TABLE 1

| | | Max Effi. | | | | Max |
|---|---|---|---|---|---|---|
| | Vₜₕ | | | | mA/ | Lumin. |
| Device structure | (V) | cd/A | cd/m² | V | cm² | (cd/m²) |
| ITO/PEDOT/MEH- | 3.0 | 0.007 | 46 | 6.0 | 652 | 96 |
| PPV/Al | | | | | | |
| ITO/PEDOT/MEH- | 2.0 | 1.33 | 3442 | 4.2 | 259 | 18552 |
| PPV/Ba/Al | | | | | | |
| ITO/PEDOT/MEH- | 1.9 | 1.69 | 2240 | 3.4 | 132 | 20801 |
| PPV/FFF-Blm₄/Al | | | | | | |

### REFERENCES

1. Huang, F.; Hou, L. T.; Wu, H. B.; Wang, X. H.; Shen, H. L.; Cao, W.; Yang, W., Cao, Y. J. Am. Chem. Soc. 2004, 126, 9845.
2. Shen, H. L.; Huang, F.; Hou, L. T.; Wu, H. B.; Cao, W.; Yang, W.; Cao, Y. Synth. Met. 2005, 152, 257.
3. Hoven, C.; Yang, R.; Garcia, A.; Heeger, A. J.; Nguyen, T.-Q.; Bazan G. C. J. Am. Chem., Soc. 2007, 129, 10976.
4. (a) Pei, Q. B.; Yang, Y.; Yu, G.; Zhang, C.; Heeger, A. J. J. Am. Chem. Soc. 1996, 118, 3922. (b) Zhang, Q. S.; Zhou, Q. G.; Cheng, Y. X.; Wang, L. X.; Ma, D. G.; Jing, X. B.; Wang, F. S. Adv. Func. Mater. 2006, 16, 1203
5. Wang, S.; Bazan, G. C. Adv. Mat 2003, 15, 1425.
6. Yang, R. Q.; Wu, H. B.; Cao, Y.; Bazan, G. C. J. Am. Chem. Soc. 2006, 128, 14422.
7. Gong, X.; Wang, S.; Moses, D.; Bazan, G. C.; Heeger, A. J. Adv. Mater. 2005, 17, 2053.
8. Ma, W. L.; Iyer, P. K.; Gong, X.; Liu, B.; Moses, D.; Bazan, G. C.; Heeger, A. J. Adv. Mater. 2005, 17, 274.
9. Steuerman, D. W.; Garcia, A.; Yang, R.; Nguyen, T.-Q., Adv. Mater 2008, 20, 528.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims.

1. An organic electronic or optoelectronic device containing a conjugated oligoelectrolyte.

2. The device of claim 1 wherein the conjugated oligoelectrolyte is the charge injection or transport layer.

3. The device of claim 1 wherein the oligoelectrolyte is a cationic conjugated oligoelectrolyte.

4. The device of claim 1 wherein the oligoelectrolyte is an anionic conjugated oligoelectrolyte.

5. The device of claim 1 wherein the conjugated oligoelectrolyte is selected from the group consisting of 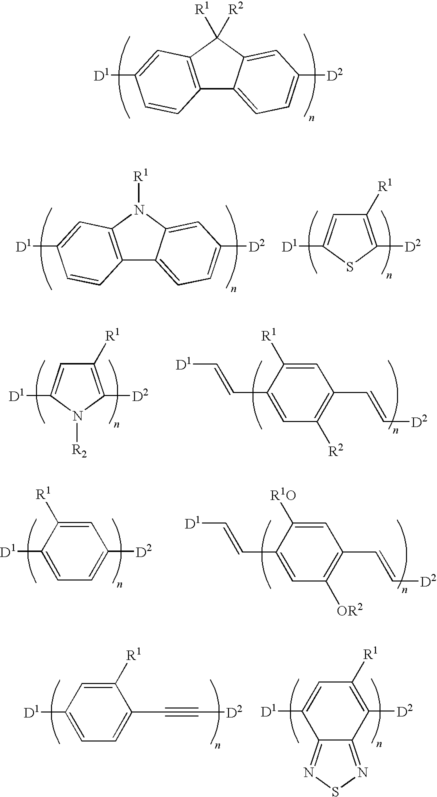
where ‘n’ is any integer between 1 and 20; and where terminal end groups D¹ and D² are the same or different and can be a hydrogen, alkyl group, alkoxy group or any one of the following: 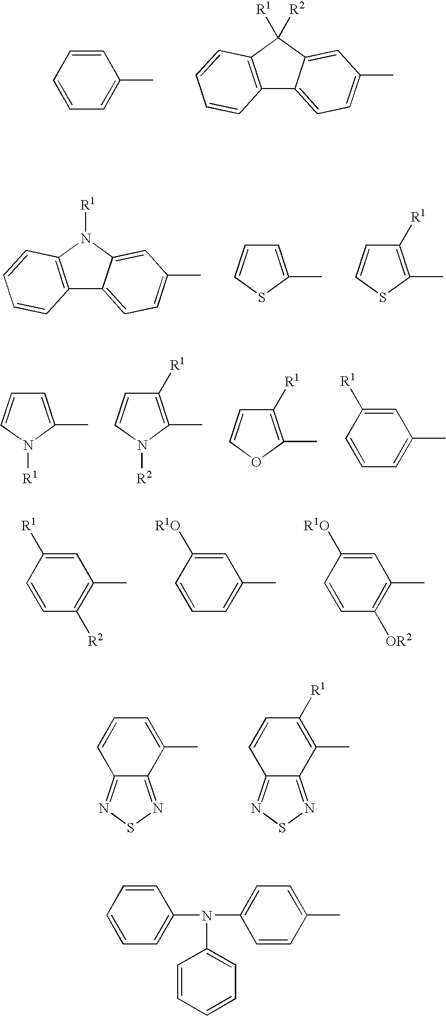
and where R¹ and R² can be the same or different and where R¹ and R² can be either:
i) (CH₂)ₙN⁺BX⁻, where ‘B’ is Me₃, Ethyl₃, Propyl₃, Me₂Ethyl, MeEthyl₂, Me₂Propyl, MePropyl₂, or Ethyl₂Propyl; ‘X’ is Br, CF₃SO₃, BIm₄, BAr^{F}₄, BF₄₁ PF₆, F₃CSO₃, BPh₄, BTh₄, BPhF₄₁ HPO₄²⁻, or FeCN₆⁴; and ‘n’ is an integer between 1 and 100; or
ii) (CH₂)ₙZ, where ‘Z’ is SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻ or (NH₄)³⁺; ‘n’ is an integer between 1 and 100.

6. The device of claim 3 wherein the cationic conjugated oligoelectrolyte any one of 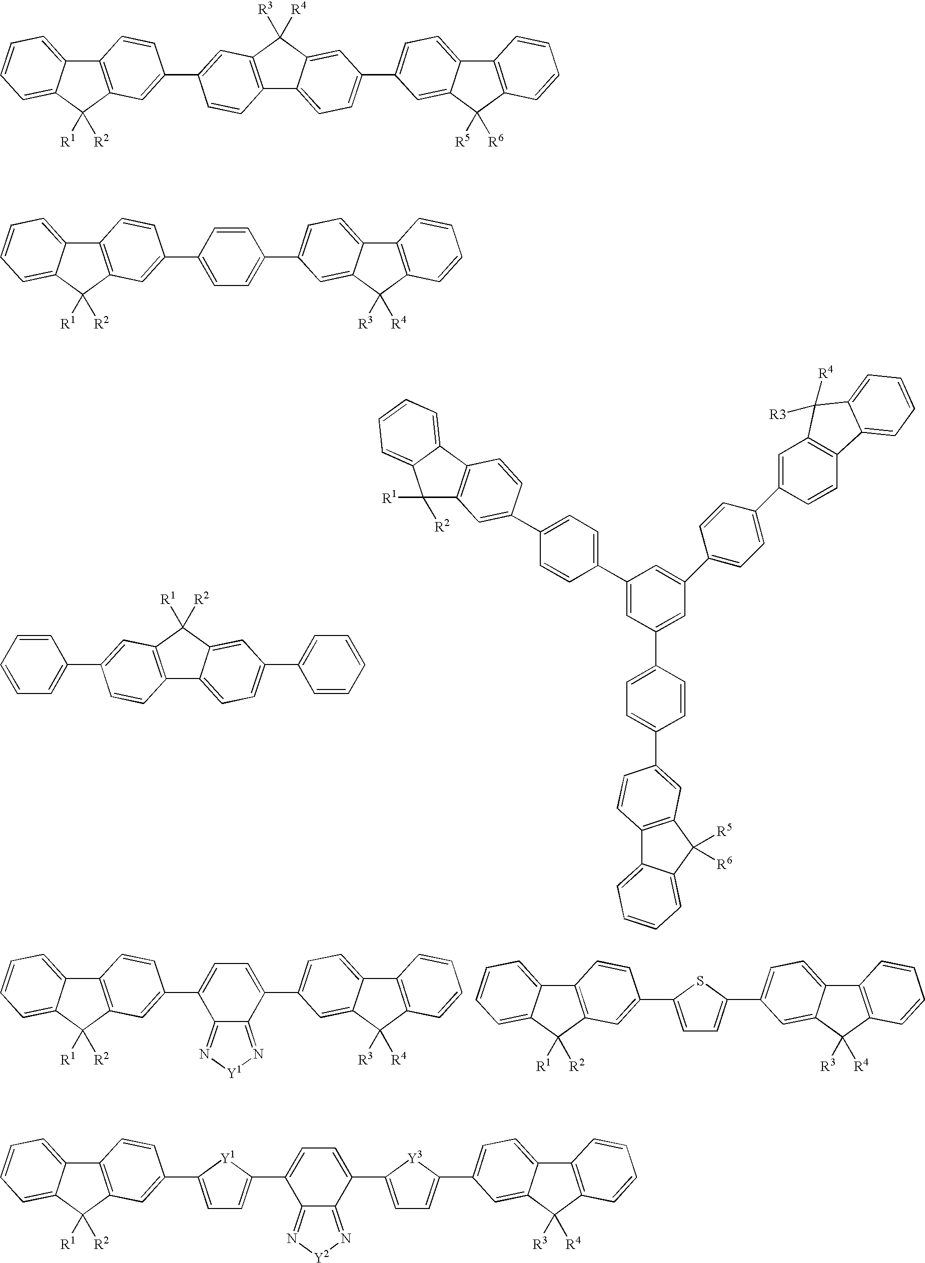 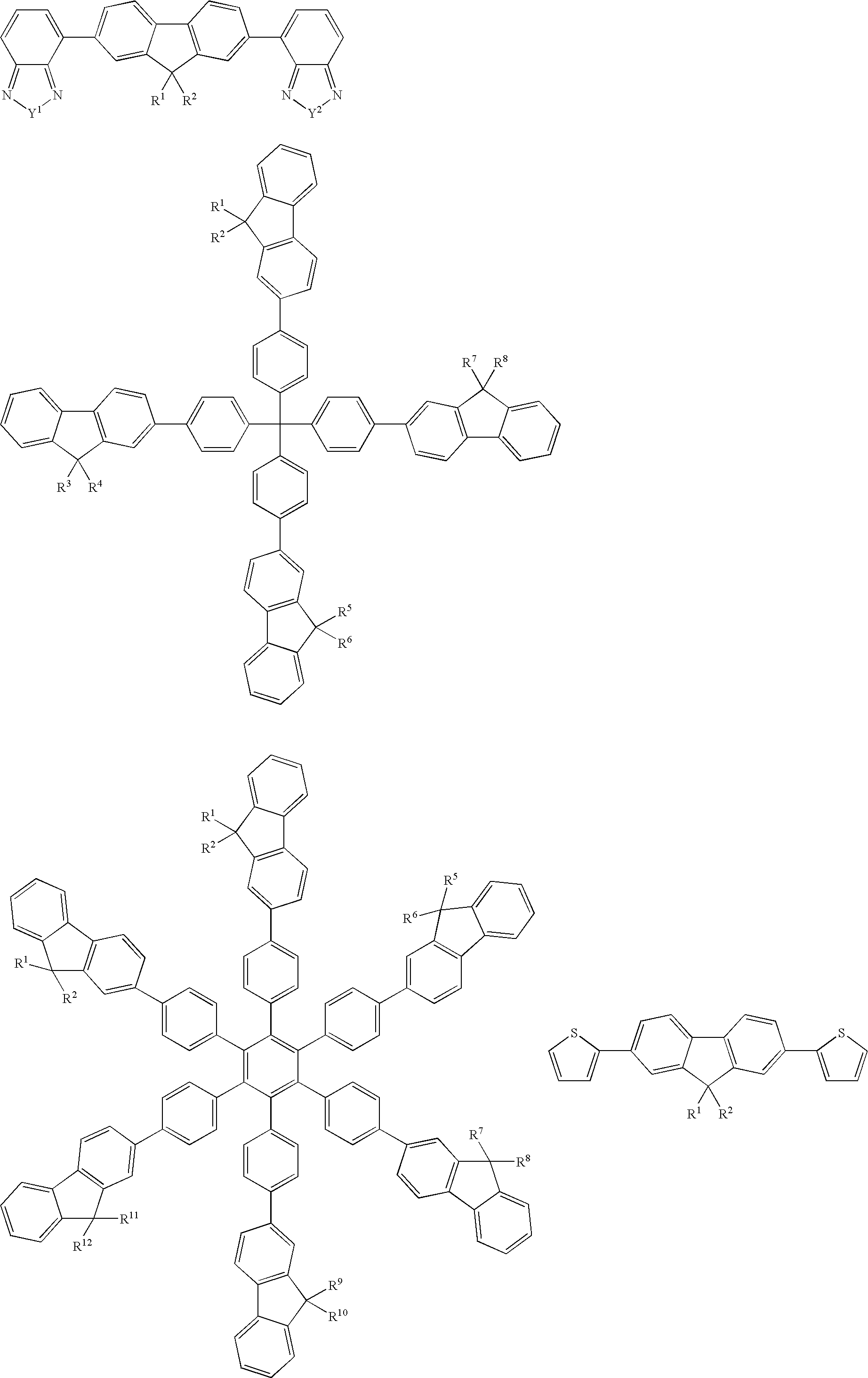 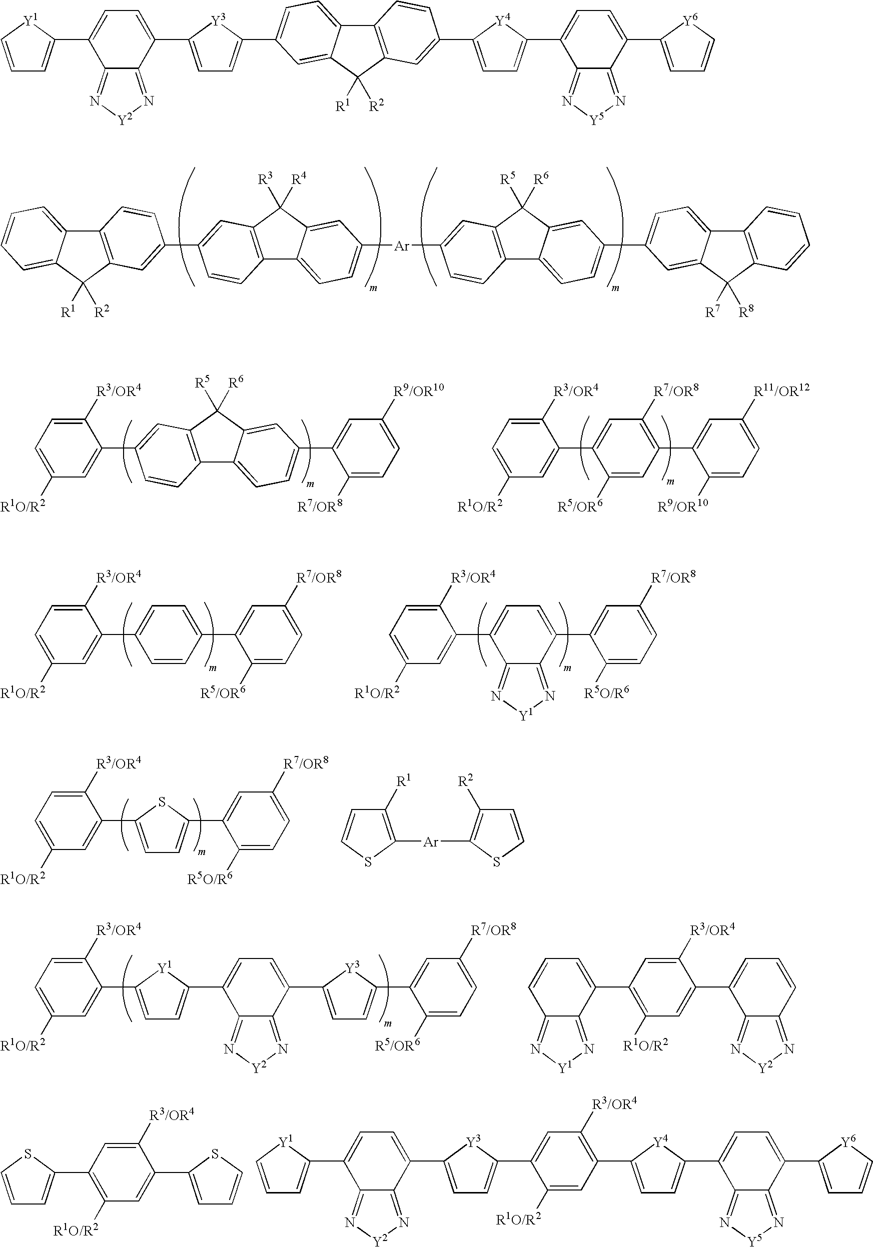 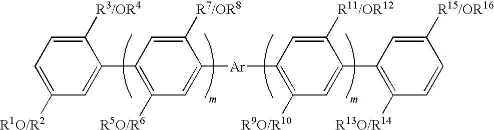
where R¹ to R¹⁶ and R′ is (CH₂)ₙN⁺BX⁻ and R¹ to R¹⁶ and R′ can be the same or different;
n is an integer between 1 and 20;
B is Me₃, Ethyl₃, Propyl₃, Me₂Ethyl, MeEthyl₂, Me₂Propyl, MePropyl₂, or Ethyl₂Propyl;
X is Br, CF₃SO₃₁ BIm₄, BArF₄, BF₄, PF₆, F₃CSO₃, BPh₄, BTh₄, BPhF₄, HPO₄²⁻, or FeCN₆⁴;
Y¹ to Y⁶ is O, S, Se, or N—R′, and where Y¹ to Y⁶ can be the same or different;
m is an integer between 1 and 20;
and wherein Ar is one of the following

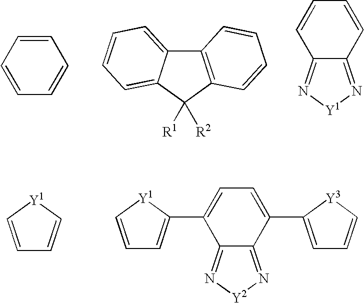

7. The device of claim 7 wherein the cationic conjugated oligoelectrolyte wherein n is 6, B is Me₃ and X is BIm₄.

8. The device of claim 7 wherein the cationic conjugated oligoelectrolyte is any one of

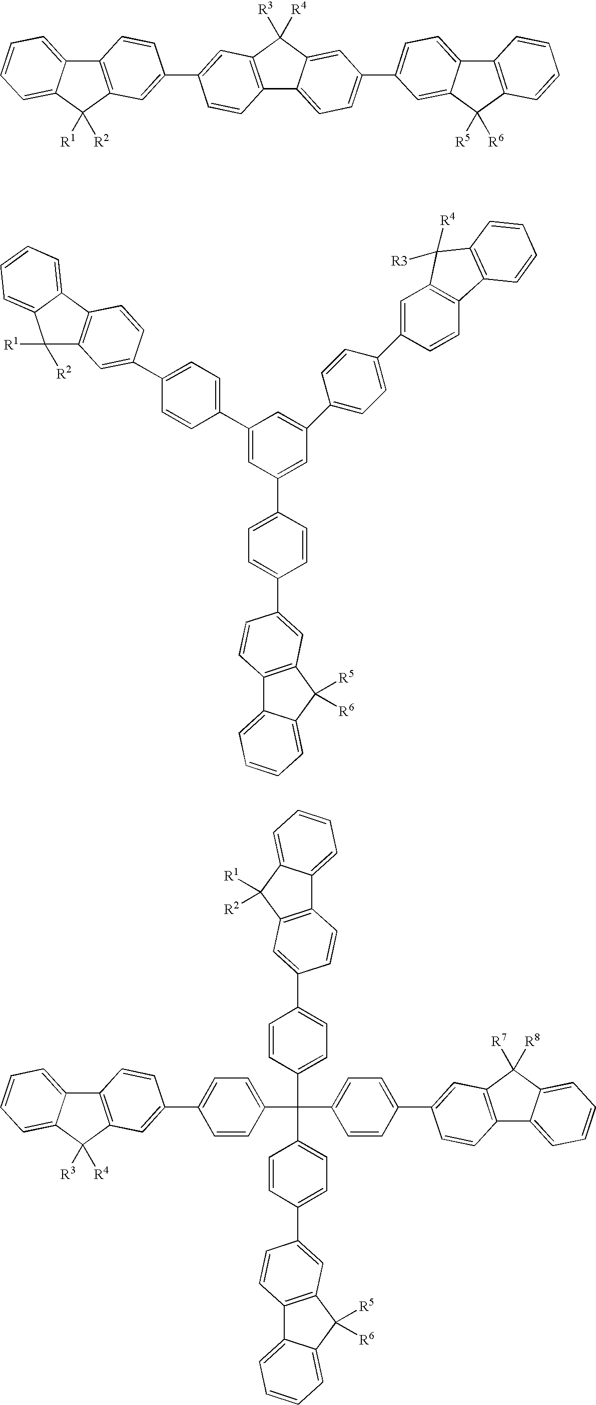

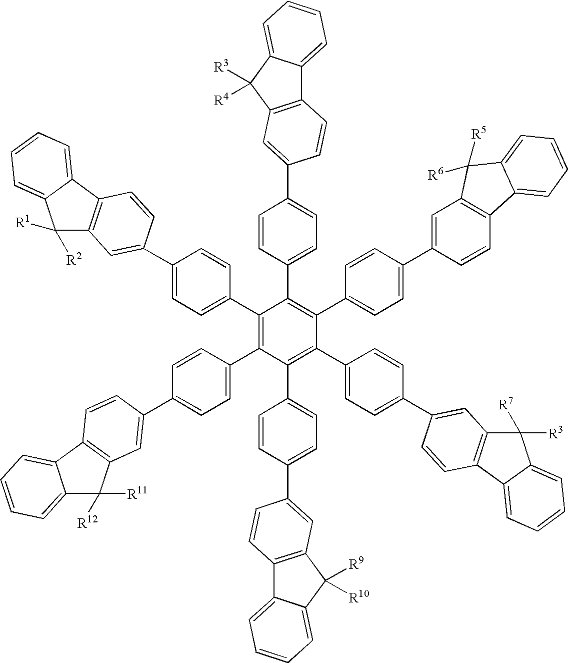

9. The device of claim 4 wherein the anionic conjugated oligoelectrolyte is any one of 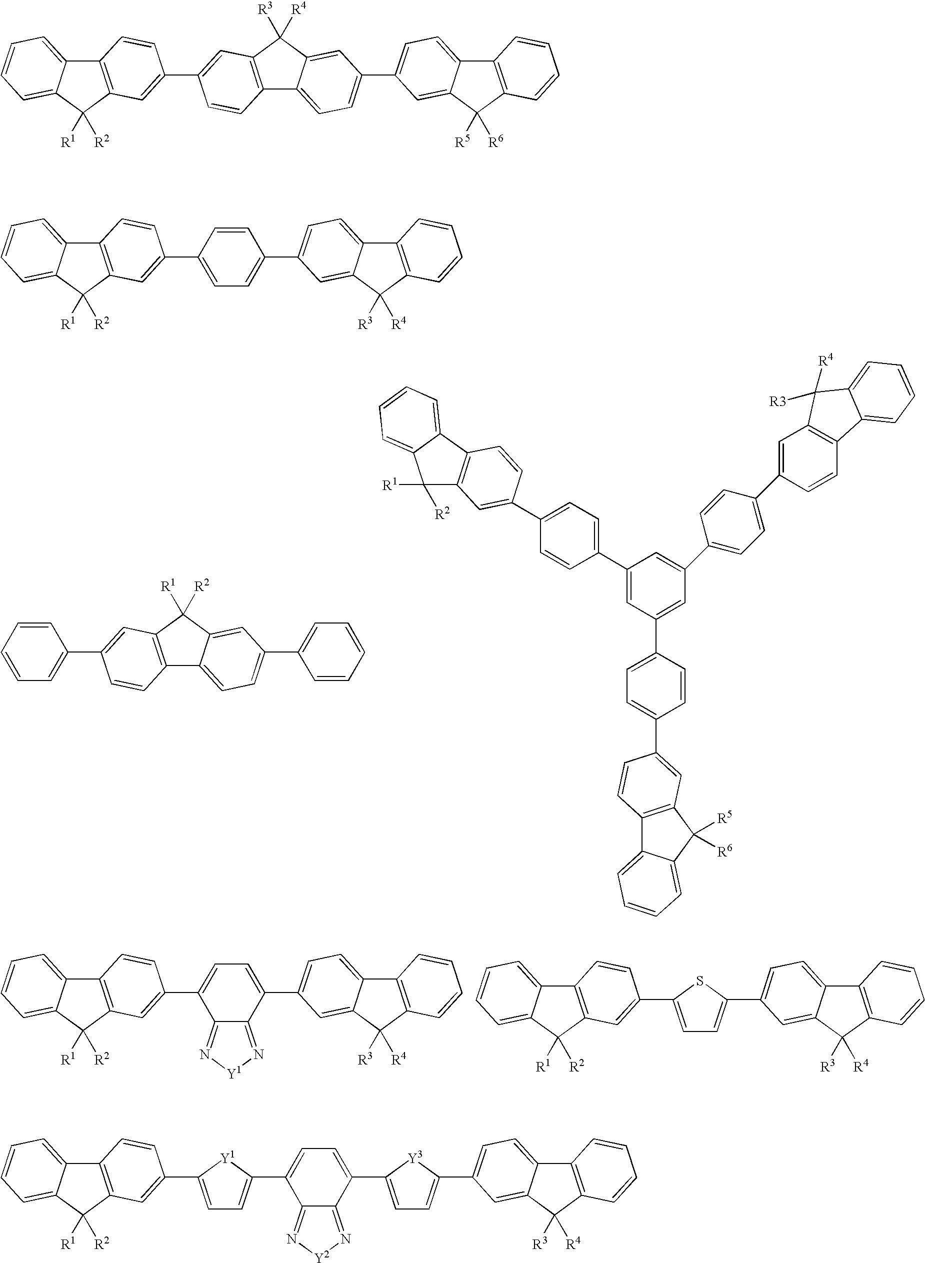 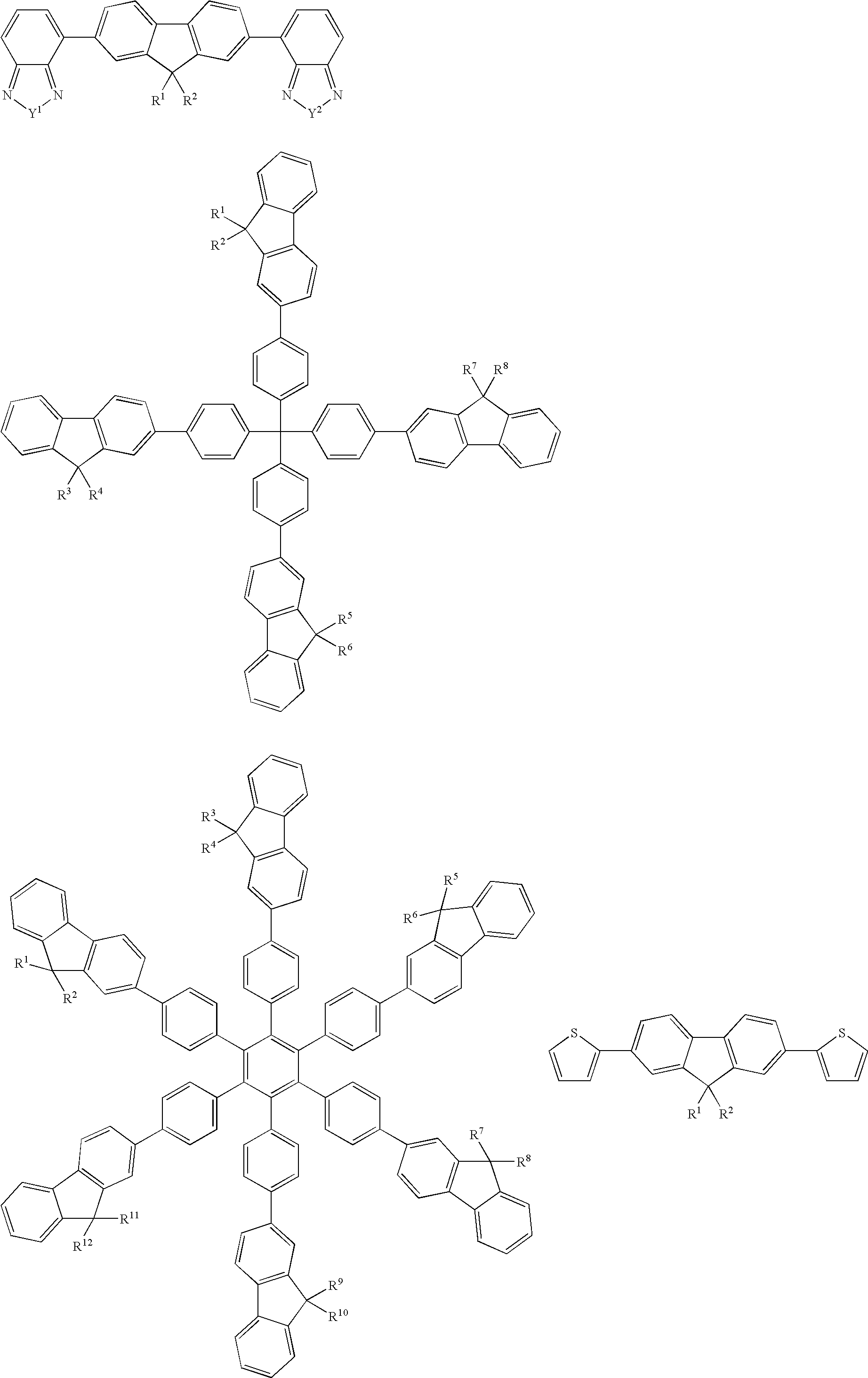 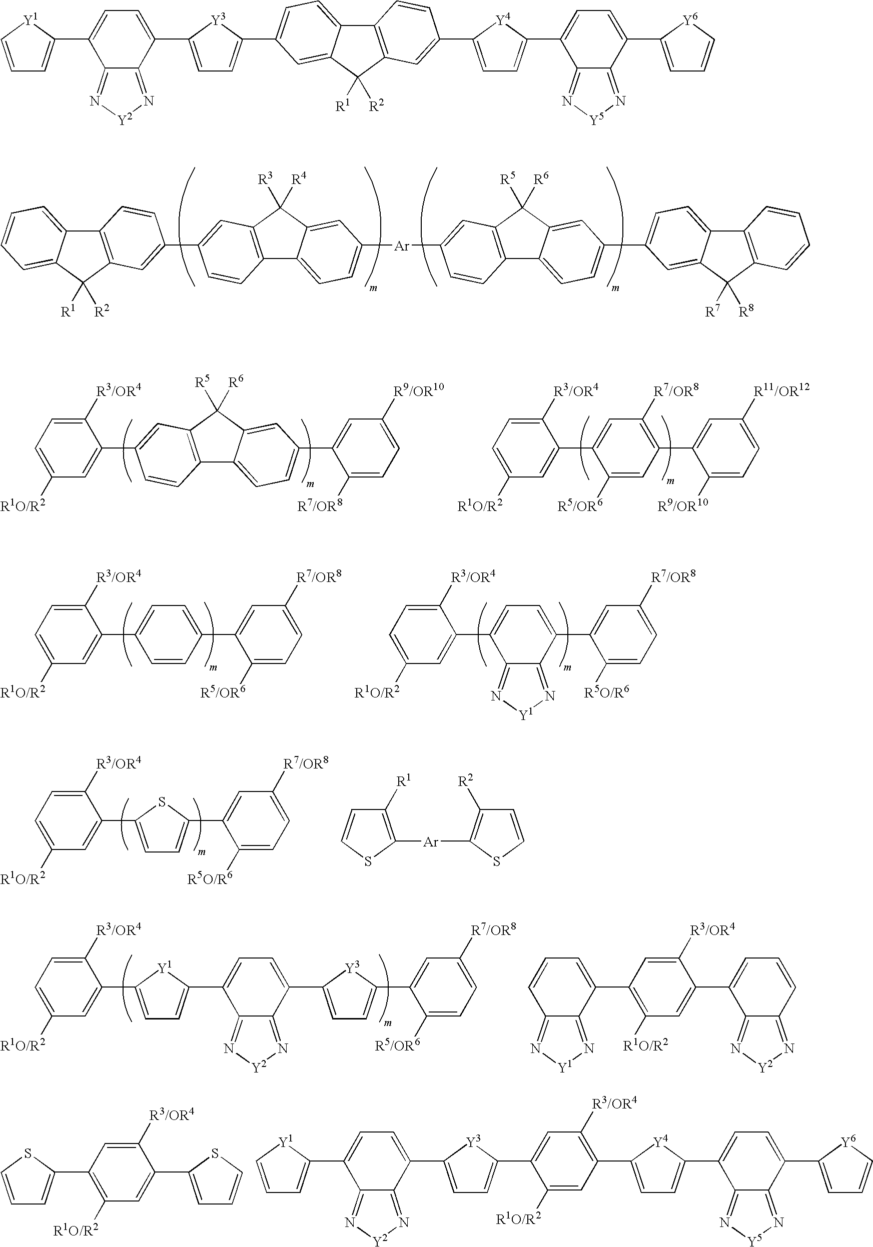 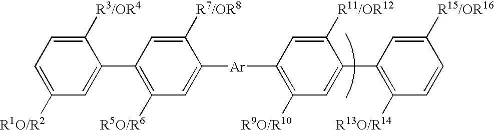
where R¹ to R¹⁶ and R′ is (CH₂)ₙZ and R¹ to R¹⁶ and R′ can be the same or different;
n is an integer between 1 and 20;
Z is SO₃⁻Na⁺, SO₃⁻K⁺, SO₃⁻NH₄⁺, COO⁻Na⁺, COO⁻K⁺, COO⁻NH₄⁺, PO₄³⁻Na₃⁺, PO₄³⁻K₃⁺, PO₄³⁻, or (NH₄)³⁺;
Y¹ to Y⁶ is O S, Se, or N—R′, and where Y¹ to Y⁶ can be the same or different;
m is an integer between 1 and 20;
and wherein Ar is one of the following

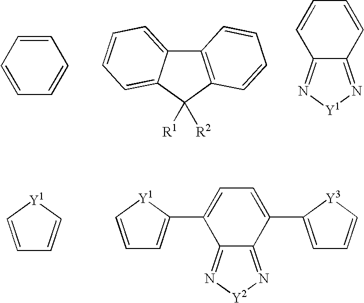

10. The device of claim 10 wherein the anionic conjugated oligoelectrolyte is any one of 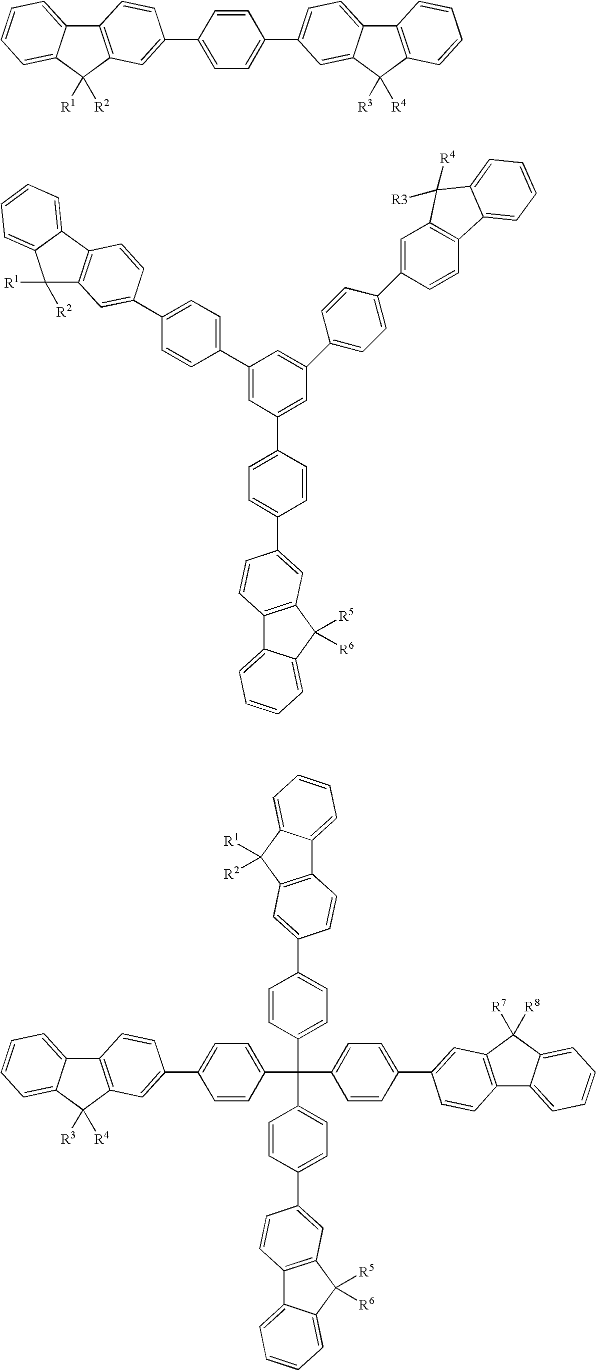 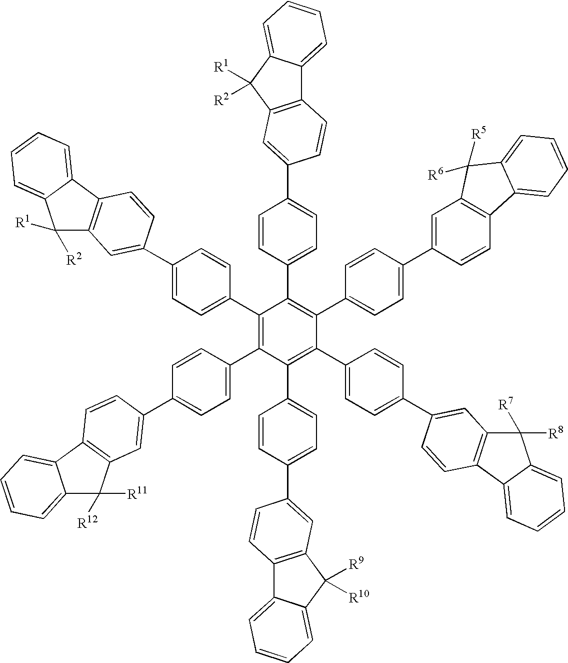
where n is 4, and Z is SO₃⁻Na⁺.

11. The organic electronic device of claim 1 further comprising a high work function metal.

12. The organic electronic device of claim 12 in which the high work function metal is Al.

13. The organic electronic device of claim 1, further comprising a low work function metal.

14. A material used for an organic electronic device which contains at least a conjugated oligoelectrolyte.

15. A method of producing an organic electronic device containing a conjugated oligoelectrolyte.
